SEQUENCE LISTING (21 sequences)

SEQ ID NO: 1 - DNA, 24 bp, Artificial Sequence
  Primer
atggacaccaccattgatggattc

SEQ ID NO: 2 - DNA, 24 bp, Artificial Sequence
  Primer
acgacgatgagagagtttacggtt

SEQ ID NO: 3 - DNA, 25 bp, Artificial Sequence
  Primer
atggtgagcaagggcgaggagctgt

SEQ ID NO: 4 - DNA, 25 bp, Artificial Sequence
  Primer
cttgtacagctcgtccatgccgaga

SEQ ID NO: 5 - DNA, 20 bp, Artificial Sequence
  Primer
tgcatcagaagcaactttgg

SEQ ID NO: 6 - DNA, 19 bp, Artificial Sequence
  Probe
cgcaaaacaagccactatg

SEQ ID NO: 7 - DNA, 20 bp, Artificial Sequence
  Primer
ggcctttgagagaatgcttg

SEQ ID NO: 8 - DNA, 20 bp, Artificial Sequence
  Primer
aactcgcctcaagactacct

SEQ ID NO: 9 - DNA, 25 bp, Artificial Sequence
  Probe
tcacaattcagctcgagcagcagtc

SEQ ID NO: 10 - DNA, 20 bp, Artificial Sequence
  Primer
tgcaactgtgtcgttccata

SEQ ID NO: 11 - DNA, 21 bp, Artificial Sequence
  Primer
acttcgctcagtaccttgttc

SEQ ID NO: 12 - DNA, 23 bp, Artificial Sequence
  Probe
atcaacagagccggccttggaaa

SEQ ID NO: 13 - DNA, 21 bp, Artificial Sequence
  Primer
ggcagtggaaaccttgatttg

SEQ ID NO: 14 - DNA, 22 bp, Artificial Sequence
  Primer
ctgtgctcggtactactgttac

SEQ ID NO: 15 - DNA, 24 bp, Artificial Sequence
  Probe
tgagaagtatcagcagcagcaggc

SEQ ID NO: 16 - DNA, 21 bp, Artificial Sequence
  Primer
cggcgatagtcatcggaatta

SEQ ID NO: 17 - DNA, 20 bp, Artificial Sequence
  Primer
tcgggtgttttcacgtctca

SEQ ID NO: 18 - DNA, 20 bp, Artificial Sequence
  Probe
tggaactcttggattttgcc

SEQ ID NO: 19 - DNA, 18 bp, Artificial Sequence
  Primer
cgccgtaggtgaggtagc

SEQ ID NO: 20 - DNA, 2104 bp, Arabidopsis thaliana
tcgatctttaaccaaatccagttgataaggtctcttcgttgattagcagagatctctttaatttgtgaatttcaattcatcggaacctgttgatggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaacgattcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcggtgagactcttgcctcttagtgtaatttttgctgtaccatataattctgttttcatgatgactgtaactgtttatgtctatcgttggcgtcatatagtttcgctcttcgttttgcatcctgtgtattattgctgcaggtgtgcttcaaacaaatgttgtaacaatttgaaccaatggtatacagatttgtaatatatatttatgtacatcaacaataaaaaaaaaaaaaaaaaa

SEQ ID NO: 21 - protein, 593 aa, Arabidopsis thaliana
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

(19) United States
(12) Patent Application Publication
(10) Pub. No.: US 2017/0073700 A1
(43) Pub. Date: Mar. 16, 2017

(54) CITRUS PLANTS RESISTANT TO HUANGLONGBING

(71) Applicant: University of Florida Research Foundation, Inc., Gainesville, FL (US)

(72) Inventors: Manjul Dutt, Winter Haven, FL (US); Jude Grosser, Winter Haven, FL (US)

(21) Appl. No.: 15/261,385

(22) Filed: Sep. 9, 2016

Related U.S. Application Data
(60) Provisional application No. 62/217,556, filed on Sep. 11, 2015.

(51) Int. Cl. C12N 15/82; A01H 4/00

(52) U.S. Cl. CPC C12N 15/8281; A01H 4/00

(57) ABSTRACT

The present invention relates to transgenic citrus trees resistant to Huanglongbing disease (HLB) through overexpression of AtNPR1 either in the phloem tissues (where HLB resides) via utilization of a phloem specific Arabidopsis sucrose-proton symporter 2 (AtSUC2) promoter or a constitutive CaMV 35S promoter for HLB resistance. Evaluation of these transgenic plants demonstrates that overexpressing the AtNPR1 can result in effective HLB resistance in citrus.

### CROSS-REFERENCE TO RELATED APPLICATIONS

This application claims the priority of U.S. Provisional Appl. Ser. No. 62/217,556, filed Sep. 11, 2015, the entire disclosure of which is incorporated herein by reference.

### INCORPORATION OF SEQUENCE LISTING

The sequence listing that is contained in the file named “UFFL074US_ST25.txt,” which is 11.3 kilobytes as measured in Microsoft Windows operating system and was created on Sep. 7, 2016, is filed electronically herewith and incorporated herein by reference.

### FIELD OF THE INVENTION

This invention relates to plant disease resistance. More specifically, the invention relates to transgenic citrus plants having increased resistance to Huanglongbing disease.

### BACKGROUND OF THE INVENTION

In the United States, Huanglongbing (HLB) is a serious disease of citrus associated with the phloem-limited bacterium Candidatus Liberibacter asiaticus (CLas). This disease is spread by the Asian citrus psyllid (ACP) vector, Diaphorina citri (Kuwayama). HLB was first detected in the United States in August 2005 and since then has rapidly moved into several citrus producing areas. Tree decline is usually preceded by a decline in the quality of the fruit and fruit drop. Fruit from infected trees are smaller, yield less juice, have higher acidity and lower sugar and peel color than fruits from uninfected trees. Infected citrus trees will irrevocably decline. Currently, HLB management consists of preventing trees from becoming infected. Prevention includes protection of the young flush from the psyllid vector or destruction of infected plant material to prevent further spread of the disease. Due to the lack of rapid curative methods to control HLB, prevention of new infections is essential in HLB management. New infections could be prevented and the disease could be managed by planting trees that are tolerant or resistant to the disease.

Utilization of resistant germplasm to slow the spread of HLB in citrus is difficult due to the lack of commercially available resistant rootstock/scion combinations that can provide a robust protection and prevent infection. Identification and incorporation of resistance traits from tolerant Citrus spp. and Citrus relatives is a potential disease management strategy. However, citrus crop improvement using conventional breeding methods is difficult and time consuming due to the long juvenile phase in citrus, which can vary from 4 to 12 years. Genetic improvement of citrus through genetic engineering remains the fastest method for improvement of an existing citrus cultivars and has been a key component for genetic improvement.

### SUMMARY OF THE INVENTION

In one aspect, the invention provides a citrus tree comprising an NPR1 coding sequence, wherein the citrus tree exhibits increased tolerance to Huanglongbing (HLB) compared to a plant not comprising said NPR1 coding sequence. In some embodiments, the NPR1 coding sequence is heterologous. In other embodiments, the NPR1 coding sequence is from Arabidopsis thaliana. In another embodiment, the citrus tree overexpresses NPR1 in the phloem tissues, or in the root tissues. In some embodiments, a citrus tree as described herein comprising an NPR1 coding sequence is resistant to HLB. In other embodiments, the citrus tree is a sweet orange tree, or the sweet orange tree is a ‘Hamlin’ or a ‘Valencia’ sweet orange tree or variety. In still further embodiments, the NPR1 coding sequence is operably linked to a heterologous promoter active in plant tissue, such as a phloem-specific promoter or a constitutive promoter. In some embodiments, the phloem-specific promoter comprises an Arabidopsis sucrose-proton symporter 2 (AtSUC2) promoter, or said constitutive promoter comprises a CaMV 35S promoter. In another embodiment, tolerance to Huanglongbing is the result of systemic acquired resistance (SAR) in the citrus tree. In another embodiment, the invention provides a seed, cell, tissue, progeny, fruit, or other plant part of a citrus tree comprising an NPR1 coding sequence, wherein the citrus tree exhibits increased tolerance or increased resistance to Huanglongbing compared to a plant not comprising said NPR1 coding sequence. In some embodiments, the plant part is selected from the group consisting of fruit, seed, tissue, stem, root, phloem, and flower. In other embodiments, the fruit, seed, tissue, stem, root, phloem, and flower comprise a heterologous NPR1 coding sequence.

In another aspect, the invention provides a method of increasing tolerance to Huanglongbing in a citrus tree that is otherwise susceptible to Huanglongbing, the method comprising expressing in the citrus tree an NPR1 coding sequence, wherein expression of said NPR1 coding sequence results in increased tolerance to Huanglongbing in the tree. In some embodiments, the NPR1 coding sequence is from Arabidopsis thaliana. In another embodiment, the citrus tree is a sweet orange tree, such as a ‘Hamlin’ or a ‘Valencia’ sweet orange tree. In another embodiment, the citrus tree comprises an NPR1 coding sequence operably linked to a heterologous promoter active in plant tissue, such as a phloem-specific promoter or a constitutive promoter. In other embodiments, the phloem-specific promoter comprises an Arabidopsis sucrose-proton symporter 2 (AtSUC2) promoter, or said constitutive promoter comprises a CaMV 35S promoter.

In another aspect, the invention provides a method of increasing yield of a citrus tree comprising expressing in the citrus tree an NPR1 coding sequence, wherein expression of said NPR1 coding sequence results in increased tolerance to Huanglongbing in the tree. In one embodiment, the NPR1 coding sequence is from Arabidopsis thaliana. In another embodiment, the transgene is operably linked to a heterologous promoter active in plant tissue, such as a phloem-specific promoter or a constitutive promoter. In another embodiment, the phloem-specific promoter comprises an Arabidopsis sucrose-proton symporter 2 (AtSUC2) promoter, or said constitutive promoter comprises a CaMV 35S promoter.

In another aspect, the invention provides a method of producing a citrus tree tolerant to HLB comprising: (a) introducing into the citrus tree an NPR1 coding sequence; and (b) reproducing the plant to produce a progeny plant. In one embodiment, the NPR1 coding sequence is from Arabidopsis thaliana. In another embodiment, step (b) comprises sexual or asexual reproduction of the plant, or asexual reproduction comprises grafting, budding, layering, or top-working. In still further embodiments. The invention provides a citrus tree produced by such a method.

In some embodiments, the present invention provides a citrus tree or variety expressing an NPR1 coding sequence in the root. In one embodiment, the NPR1 coding sequence is from Arabidopsis thaliana. Citrus roots expressing NPR1 may be used as a rootstock onto which a second citrus plant or variety may be grafted. In some embodiments, the scion or citrus variety grafted onto the rootstock may exhibit resistance or tolerance to HLB as a result of the grafting. In other embodiments, a viral vector, such as a viral vector based on a Citrus Tristeza Virus may be used to introduce and/or express an NPR1 coding sequence in a citrus rootstock or a scion as described herein.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1—Shows the process of systemic acquired resistance (SAR) induction in citrus.

FIGS. 2A and 2B—Shows a schematic representation of the T-DNA region of the binary vectors used in this study. (A) Phloem targeted gene construct. (B) Constitutive expression gene construct. CaMV, double enhanced (2×−343 to −90) CaMV 35S promoter; dCsVMV, double enhanced (2×−443 to −123) CsVMV promoter; AtSUC2, The Arabidopsis sucrose synthase promoter, egfplnptII, bifunctional enhanced green fluorescent protein and neomycin phosphotransferase II fusion gene; NOS-3′, termination site and polyadenylation signal of the NOS transcript; 35S-3′, termination site and polyadenylation signal of the CaMV 35S transcript; RB, right border; LB, left border. The arrow represents the direction of transcription.

FIG. 3A-3F—Shows (A) a set of transgenic trees with the AtNPR1 construct; (B) Close-up of an HLB-positive transgenic tree with the AtNPR1 construct; (C) A heavily infected HLB-positive control tree; (D) 2-foot spacing between two adjacent trees in the field plot. Normal citrus trees are usually planted at an 8-foot spacing or greater, (E) Close-up of a healthy flush; (F) Close-up of an HLB-infected leaf.

FIG. 4—Shows duplex PCR amplification products of the AtNPR1 and egfp genes from genomic DNA of transgenic sweet orange citrus plants. Transgenic lines 1 to 7 are 35S-NPR1 lines, while lines 8 to 14 are AtSUC2-NPR1 lines. Amplification was carried out using gene specific primers which gave the expected 1.8 kb AtNPR1 fragment and 0.7 egfp fragment (arrows). M, 1 kb DNA ladder; 1-14 are 14 randomly selected individual transgenic lines.

FIGS. 5A and 5B—Shows quantification of AtNPR1 activity using qPCR. Total RNA extracted from an entire sweet orange leaf (A) or specifically midrib and petioles (B) was used as template. Sequence of primers used to amplify the AtNPR1 gene is detailed in Table 1. Transgenic lines 1 to 16 containing the 35S-NPR1 cassette are ‘Hamlin’, while lines 17 to 31 are ‘Valencia’. Transgenic lines 1 to 12 containing the AtSUC2-NPR1 cassette are ‘Hamlin’, while lines 13 to 19 are ‘Valencia’. Three independent clones were tested from each transgenic line. Total RNA from a non-transgenic plant was also included to verify the accuracy of the amplification process. Transgenic plants that had a level of expression greater than indicated by the dotted line were considered to exhibit a relative high level of expression of AtNPR1.

FIG. 6—Shows Southern hybridization analysis of total DNA from leaf tissue of five AtNPR1 transformed sweet orange lines (2, 4, 9A, 11A, 18) and a non-transgenic control plant. Lines denoted with a number are constitutively expressing lines, while lines with the suffix ‘A’ are phloem-specific lines.

FIG. 7—Shows quantification of gene activity using qPCR. Sequence of primers used in the qPCR process is detailed in Table 1. Three independent clones were tested from each transgenic line. Total RNA from a non-transgenic plant was also included to verify the accuracy of the amplification process. Selected data from FIG. 4 (AtNPR1) is included for comparison.

FIG. 8—Shows survival of transgenic plants and control after 1 and 2 years in a no-choice greenhouse evaluation study and exposed to free flying potentially CLas containing psyllids.

### BRIEF DESCRIPTION OF THE SEQUENCES

SEQ ID NO:1—NP51 primer for amplification of AtNPR1 transgene in transgenic citrus plants.

SEQ ID NO:2—NP32 primer for amplification of AtNPR1 transgene in transgenic citrus plants.

SEQ ID NO:3—EG-51 primer for amplification of egfp transgene in transgenic citrus plants.

SEQ ID NO:4—EG-32 primer for amplification of egfp transgene in transgenic citrus plants.

SEQ ID NO:5-7—Primer/probe sequences used in real-time PCR assay for detection of AtNPR1 gene of transgenic citrus plants.

SEQ ID NO:8-10—Primer/probe sequences used in real-time PCR assay for detection of CsPR1 gene of transgenic citrus plants.

SEQ ID NO:11-13—Primer/probe sequences used in real-time PCR assay for detection of CsPR2 gene of transgenic citrus plants.

SEQ ID NO:14-16—Primer/probe sequences used in real-time PCR assay for detection of CsWRKY70 gene of transgenic citrus plants.

SEQ ID NO:17-19—Primer/probe sequences used in real-time PCR assay for detection of 18s rRNA gene of transgenic citrus plants.

SEQ ID NO:20—Coding sequence of Arabidopsis thaliana NPR1 mRNA.

SEQ ID NO:21—Protein sequence of Arabidopsis thaliana NPR1.

### DETAILED DESCRIPTION OF THE INVENTION

The invention provides a citrus tree comprising an NPR1 gene, wherein the citrus tree exhibits increased tolerance to Huanglongbing (HLB) compared to a plant not comprising said NPR1 gene. Also provided are seeds, fruit, and plant parts of such citrus trees, along with methods for producing citrus trees that are tolerant to HLB. Also provided are methods of increasing the yield of a citrus tree comprising expressing an NPR1 coding sequence in the tree, wherein expression of said NPR1 coding sequence results in increased tolerance to Huanglongbing in the tree. In other embodiments, methods are provided for producing a citrus tree tolerant to HLB comprising: (a) introducing into the citrus tree an NPR1 coding sequence; and (b) reproducing the plant to produce a progeny plant. Such methods may comprise sexual or asexual reproduction of the plant, including grafting or any other methods appropriate for use with citrus.

The invention provides a significant improvement over the art because all currently available commercial sweet orange cultivars lack resistance to HLB. HLB is a serious phloem-limited bacterial disease that is usually fatal to affected trees. In order to develop sustained disease resistance to HLB, transgenic sweet orange cultivars ‘Hamlin’ and ‘Valencia’ expressing an Arabidopsis thaliana NPR1 gene under the control of a constitutive CaMV 35S promoter or a phloem-specific Arabidopsis SUC2 (AtSUC2) promoter were produced. Overexpression of AtNPR1 resulted in trees with normal phenotypes that exhibited enhanced resistance to HLB. Phloem-specific expression of NPR1 was equally effective for enhancing disease resistance. Transgenic trees exhibited reduced diseased severity and a few lines remained disease-free even after 36 months of planting in a high disease-pressure field site. Expression of the NPR1 gene induced expression of several native genes involved in the plant defense signaling pathways. The AtNPR1 gene being plant derived can serve as a component for the development of an all-plant T-DNA derived consumer friendly genetically modified tree.

HLB, a phloem-restricted bacterial disease of citrus has been present in the United States since 2005 [33]. This disease has resulted in a severe decline in fruit production in Florida, where it has become endemic [34]. Florida produces sweet oranges, predominantly for juice production and all commercial cultivars are susceptible to HLB [35]. Development of new cultivars through conventional hybridization is very difficult due to the high level of nuclear embryony in these cultivars. All major commercial sweet orange cultivars have arisen through the development of mutations and have been subsequently selected over hundreds of years [36]. In such cases, genetic improvement of existing cultivars without otherwise changing its characteristics through the incorporation of an additional advantageous trait remains the fastest method of improvement.

Genetic engineering of sweet oranges is a viable alternative to conventional breeding as it is a relatively rapid process and it allows for the insertion of a single trait without modifying existing traits. In this study, ‘Hamlin’ and ‘Valencia’ sweet oranges were transformed with the AtNPR1 transgene via Agrobacterium-mediated genetic transformation to produce transgenic plants tolerant to HLB.

NPR1 is a key regulator of gene expression following infection [37] and controls the onset of the immune response known as systemic acquired resistance (SAR) [38]. AtNPR1 has been directly implicated for fungal disease resistance in wheat [39], cotton [40], broad spectrum disease resistance in strawberry [41], tomato [42], carrot [43], and bacterial disease resistance in citrus [11]. In addition, AtNPR1 homologs have been identified in many economically important plants such as citrus [44], gladiolus [45], grapevine [46], rice [47], phalaenopsis orchid [48], and sugarbeet [49], among others. Development of HLB-resistant citrus by exploiting the plants own immune system is a potentially attractive approach to develop a genetically modified consumer-acceptable plant. This strategy utilizes a transgene whose homolog is available in many food crops, including citrus.

Regenerated trees exhibited normal phenotypes and did not demonstrate the abnormalities that were observed in strawberry plants constitutively expressing AtNPR1 [41] or the rice AtNPR1 homolog (NH1) in rice [47]. Homology-dependent gene silencing can be an issue when endogenous genes are overexpressed in the same system [50, 51], which led to experiments aimed at overexpressing the Arabidopsis thaliana NPR1 gene (AtNPR1) in citrus plants, rather than the citrus homolog. The results indicate that overexpression of the AtNPR1 gene can induce resistance to HLB with a reduced disease severity phenotype in many lines. Resistance could not be directly co-related to AtNPR1 gene expression levels, as several transgenic lines with good AtNPR1 expression levels were susceptible to HLB. This could be due to differential insertion of the transgene cassette in the individual lines. AtNPR1 produced either constitutively or in the phloem was observed to be sufficient in combatting HLB. Since NPR1 regulates the signal transduction pathway that results in SAR [18], gene expression in the phloem cells was sufficient to induce the PR genes resulting in disease resistance. Molecular analyses revealed the presence of the coding sequence of the introduced AtNPR1 and expression of the gene in transgenic sweet orange plants. Analyzed lines had less than 3 copies of the transgene stably incorporated into the genome. It was previously observed that an increase in copy number negatively affected the gene expression in citrus [52], and current results support that observation. Three genes involved in plant defense signaling pathways, PR1 [37, 53], PR2 [11, 54], and WRKY70 [55, 56] were evaluated in this study based on their ability to be differentially regulated by AtNPR1. AtNPR1 induces PR1 gene expression [31], and the single copy insert (transgenic line 2) had both the highest NPR1 expression and PR1 expression. In fact, levels of PR1 gene expression could be directly co-related to the transgene mediated resistance to HLB. PR2, which has been observed to be directly responsible for the SAR process [11, 57] was also overexpressed in all the transgenic lines, although not at the levels observed for PR1 expression. The WRKY70 transcription factor influences the defense pathways [58], and specifically the salicylate-mediated signaling pathways in plant defense [56]. Apart from line 18, all other lines behaved similarly, demonstrating the activation of the SAR pathways. The observed results are contradictory to that observed previously in citrus [11] where constitutive defense responses were not observed following overexpression of AtNPR1. A few of the transformed lines did not exhibit enhanced gene expression indicating post-transcriptional gene silencing or inefficient nicking of T-DNA borders and co-transfer of non-T-DNA sequences into the citrus genome[59,60]. The basic mechanism behind SAR is generally conserved across species, but based on the results described herein and in the Examples, it is apparent that there is a differential gene expression pattern following SAR between citrus and other crop plants

The embodiments described herein are not limited to a particular citrus tree or variety but rather any citrus variety or hybrid thereof may be useful in accordance with the invention, including but not limited to, sweet orange, bitter orange, blood orange, grapefruit, pomelo, citron, clementine, naval orange, lemon, lime, mandarin, tangerine, tangelo, or the like. In some embodiments, the citrus tree may be a sweet orange tree, such as a ‘Hamlin’ or a ‘Valencia’ sweet orange tree.

In the United States, Huanglongbing (HLB) is a serious disease of citrus associated with the phloem-limited bacterium Candidatus Liberibacter asiaticus (CLas) [1]. This disease is spread by the Asian citrus psyllid (ACP) vector, Diaphorina citri (Kuwayama) [2]. HLB was first detected in the United States in August 2005 and since then has rapidly moved into several citrus producing areas [3, 4]. Tree decline is usually preceded by a decline in the quality of the fruit and fruit drop. Fruit from infected trees are smaller, yield less juice, have higher acidity and lower sugar and peel color than fruits from uninfected trees [5]. Infected citrus trees will irrevocably decline. Currently, HLB management consists of preventing trees from becoming infected [4].

Prevention includes protection of the young flush from the psyllid vector [6] or destruction of infected plant material to prevent further spread of the disease. Due to the lack of rapid curative methods to control HLB, prevention of new infections is essential in HLB management [7]. New infections could be prevented, and the disease could be managed, by planting trees that are tolerant or resistant to the disease [6].

Utilization of resistant germplasm to slow the spread of HLB in citrus is difficult due to the lack of commercially available resistant rootstock/scion combinations that can provide a robust protection and prevent infection. Identification and incorporation of resistance traits from tolerant Citrus spp. and Citrus relatives is a potential disease management strategy [8]. However, citrus crop improvement using conventional breeding methods is difficult and time consuming due to the long juvenile phase in citrus, which can vary from 4 to 12 years [9]. Genetic improvement of citrus through genetic engineering remains the fastest method for improvement of an existing citrus cultivars and has been a key component in the University of Florida's genetic improvement strategy [10].

Genetic improvement of citrus using genes that allow plants to defend themselves against pathogens utilizing systemic acquired resistance (SAR) has resulted in the production of transgenic canker resistant trees [11]. SAR is a plant defense response resulting in the expression of specific defense genes that enhances innate resistance to further infection by pathogens [12].

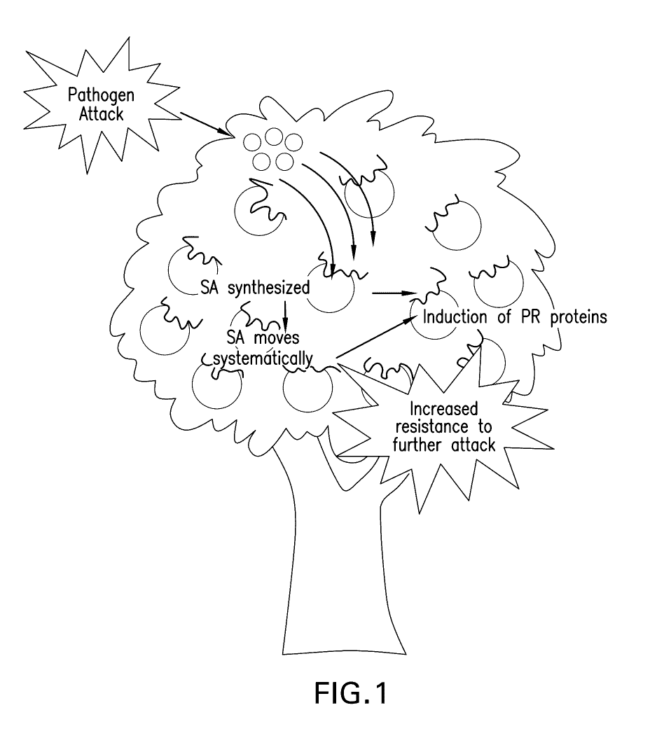

Utilization of SAR is a novel method to employ the plant's inherent immune system to reduce disease development and spread (FIG. 1). SAR provides protection against a broad spectrum of microorganisms and is associated with the production of pathogenesis-related (PR) proteins [13]. This defense response is induced by salicylic acid (SA) [14], since plants that fail to produce salicylic acid also fail to develop SAR, nor do they express pathogenesis-related (PR) genes in the uninoculated leaves [15]. These plants are also more susceptible to pathogen infection [16]. For example transgenic Arabidopsis plants overexpressing the nahG gene encoding the SA hydroxylase enzyme are unable to accumulate SA due to its degradation by the SA hydroxylase enzyme into catechol. Such plants are very susceptible to infection by Pseudomonas syringae and Peronospora parasitica. Several Arabidopsis mutants which are salicylic acid induction-deficient are unable to accumulate SA after pathogen inoculation and are very susceptible to pathogens [17].

Non-expressor of Pathogenesis Related genes 1 (NPR1) gene is a key regulator in the signal transduction pathway that leads to SAR since the npr1 mutant in Arabidopsis fails to respond to various SAR-inducing agents and exhibits very low expression of several PR genes. The NPR1 gene may act as a regulator of the transcription factor/s that controls PR gene expression [18] and mediates the salicylic acid induced expression of PR genes and SAR [19]. Plants over expressing NPR1 exhibit enhanced resistance to several pathogens [20].

As described herein, transgenic sweet orange trees may be produced in which AtNPR1 is overexpressed either in the phloem tissues (where HLB resides) via utilization of a phloem-specific Arabidopsis sucrose-proton symporter 2 (AtSUC2) promoter or a constitutive CaMV 35S promoter for HLB resistance. Evaluation of these transgenic plants demonstrates that overexpressing the AtNPR1 can result in effective HLB resistance in citrus.

### I. PLANT TRANSFORMATION CONSTRUCTS

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. In some embodiments, a viral vector based on a plant virus such as a Citrus Tristeza Virus may be used in accordance with the invention. It is contemplated that utilization of cloning systems with large insert capacities will allow introduction of large DNA sequences comprising more than one selected gene. In accordance with the invention, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes that have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes.

In accordance with the invention, a nucleic acid vector comprising an NPR1 coding sequence may be introduced into a plant such as a citrus tree or variety, such that, when the vector is transformed into a citrus variety or plant as described herein, the coding sequence is expressed in the plant. In some embodiments the NPR1 coding sequence may be expressed in, for example, the phloem or roots of the plant, or any other part of the plant. Expression of the NPR1 coding sequence in the resulting transgenic citrus tree or variety results in the tree exhibiting increased tolerance or resistance to HLB when compared to a tree lacking expression of the NPR1 coding sequence.

A. Proteins and Recombinant DNA Molecules

As used herein, a “protein-coding DNA molecule” or “polypeptide-coding DNA molecule” refers to a DNA molecule comprising a nucleotide sequence that encodes a protein or polypeptide. A “coding sequence” or “protein-coding sequence” or “polypeptide-coding sequence” means a DNA sequence that encodes a protein or polypeptide. A “sequence” means a sequential arrangement of nucleotides or amino acids. The boundaries of a protein-coding sequence or polypeptide-coding sequence are usually determined by a translation start codon at the 5′-terminus and a translation stop codon at the 3′-terminus. A protein-coding molecule or polypeptide-coding molecule may comprise a DNA sequence encoding a protein or polypeptide sequence. As used herein, “transgene expression,” “expressing a transgene,” “protein expression,” “polypeptide expression,” “expressing a protein,” and “expressing a polypeptide” mean the production of a protein or polypeptide through the process of transcribing a DNA molecule into messenger RNA (mRNA) and translating the mRNA into polypeptide chains, which may be ultimately folded into proteins. A protein-coding DNA molecule or polypeptide-coding DNA molecule may be operably linked to a heterologous promoter in a DNA construct for use in expressing the protein or polypeptide in a cell transformed with the recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a protein-coding DNA molecule or polypeptide-coding DNA molecule in a DNA construct where the two DNA molecules are so arranged that the promoter may affect the expression of the transgene.

As used herein, a “DNA construct” is a recombinant DNA molecule comprising two or more heterologous DNA sequences. DNA constructs are useful for transgene expression and may be comprised in vectors and plasmids. DNA constructs may be used in vectors for the purpose of transformation, that is the introduction of heterologous DNA into a host cell, in order to produce transgenic plants and cells, and as such may also be contained in the plastid DNA or genomic DNA of a transgenic plant, seed, cell, or plant part. As used herein, a “vector” means any recombinant DNA molecule that may be used for the purpose of plant transformation. Recombinant DNA molecules as set forth in the sequence listing, can, for example, be inserted into a vector as part of a construct having the recombinant DNA molecule operably linked to a promoter that functions in a plant to drive expression of the protein encoded by the recombinant DNA molecule. Methods for constructing DNA constructs and vectors are well known in the art. The components for a DNA construct, or a vector comprising a DNA construct, generally include, but are not limited to, one or more of the following: a suitable promoter for the expression of an operably linked DNA, an operably linked protein-coding DNA molecule, and a 3′ untranslated region (3′-UTR). Promoters useful in practicing the present invention include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional optional components include, but are not limited to, one or more of the following elements: 5′-UTR, enhancer, leader, cis-acting element, intron, chloroplast transit peptides (CTP), and one or more selectable marker transgenes.

Recombinant DNA molecules of the present invention may be synthesized and modified by methods known in the art, either completely or in part, especially where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present invention includes recombinant DNA molecules and proteins having at least about 80% (percent) sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to an NPR1 coding sequence provided herein, for instance the NPR1 sequences set forth as SEQ ID NOs:20-21. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

Proteins in accordance with the invention may be produced by changing (that is, modifying) a wild-type protein to produce a new protein with a novel combination of useful protein characteristics, such as altered Vmax, Km, substrate specificity, substrate selectivity, and protein stability. Modifications may be made at specific amino acid positions in a protein and may be a substitution of the amino acid found at that position in nature (that is, in the wild-type protein) with a different amino acid. Proteins provided by the invention thus provide a new protein with one or more altered protein characteristics relative to the wild-type protein found in nature. In one embodiment of the invention, a protein may have altered protein characteristics such as improved or decreased activity against one or more herbicides or improved protein stability as compared to a similar wild-type protein, or any combination of such characteristics. In one embodiment, the invention provides an NPR1 protein, and the DNA molecule or coding sequence encoding it, having at least about 80% sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to a protein sequence such as set forth as SEQ ID NO:21. Amino acid mutations may be made as a single amino acid substitution in the protein or in combination with one or more other mutation(s), such as one or more other amino acid substitution(s), deletions, or additions. Mutations may be made as described herein or by any other method known to those of skill in the art.

B. Regulatory Elements

The plants and methods of the present invention can utilize a vector comprising an NPR1 coding sequence that, when the vector is transfected into a plant, the NPR1 coding sequence is expressed in the plant. The site and conditions under which the first selected DNA is expressed can be controlled to a great extent by selecting a promoter element in the vector that causes expression under the desired conditions.

In some embodiments, the NPR1 coding sequence is expressed primarily in the roots of the plant, or in the phloem tissue of the plant. In this case, the NPR1 coding sequence may be expressed in a greater quantity in roots or phloem than in other tissues of the plant. In some embodiments, more than one copy of an NPR1 coding sequence may be expressed in a plant such that expression in the roots or phloem may be at least twice as much as in any other individual plant tissue (e.g., leaves, flowers, etc).

Limiting expression of the NPR1 coding sequence primarily to the roots or phloem of a plant may be accomplished by operably linking the NPR1 coding sequence to a heterologous promoter active in plant tissues, such as a root-specific or phloem-specific promoter. In other embodiments, a constitutive promoter may be preferred such that the NPR1 coding sequence is expressed in all tissues of the plant. In some embodiments, a phloem-specific promoter in accordance with the invention may comprise an Arabidopsis sucrose-proton symporter 2 (AtSUC2) promoter, or a constitutive promoter may comprise a CaMV 35S promoter. Any root-specific or phloem-specific promoter known in the art may potentially be utilized to direct expression of the NPR1 coding sequence to the roots or the phloem tissue. Examples of these may include, but are not limited to, an RB7, RPE15, RPE14, RPE19, RPE29, RPE60, RPE2, RPE39, RPE61, SHR, ELG3, EXP7, EXP18 or Atlg73160 promoter (Vijaybhaskar et al., 2008; Kurata et al., 2005; PCT Publication WO 01/53502; U.S. Pat. No. 5,459,252; Cho and Cosgrove, 2002).

In some embodiments, an NPR1 coding sequence as described herein may be expressed at any level in the plant such that it may be detected in the plant using techniques known in the art. An NPR1 coding sequence may be expressed in a greater quantity in a transgenic citrus tree or variety than in a plant not expressing an NPR1 coding sequence as described herein. In some embodiments, the NPR1 coding sequence is expressed at least twice as much as in a plant not expressing an NPR1 coding sequence. In further embodiments, the NPR1 coding sequence is expressed at least three, or four, or five times, or more, as much as in a plant not expressing an NPR1 coding sequence. In yet another embodiment, there is no detectable expression of the NPR1 coding sequence in a plant not expressing an NPR1 coding sequence.

The DNA sequence between the transcription initiation site and the start of the coding sequence, i.e., the untranslated leader sequence, can also influence gene expression. One may thus wish to employ a particular leader sequence with a transformation construct of the invention. Useful leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure.

It is contemplated that vectors for use in accordance with the present invention may be constructed to include an ocs enhancer element. This element was first identified as a 16-bp palindromic enhancer from the octopine synthase (ocs) gene of Agrobacterium (Ellis et al., 1987), and is present in at least 10 other promoters (Bouchez et al., 1989). The use of an enhancer element, such as the ocs element and particularly multiple copies of the element, may act to increase the level of transcription from adjacent promoters when applied in the context of plant transformation.

C. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of an NPR1 coding sequence coding sequence may be used. Alternatively, a heterologous 3′ end may enhance the expression of NPR1 coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

D. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, Golgi apparatus, and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

E. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Many examples of suitable marker proteins are known to the art and can be employed in the practice of the invention. Examples include, but not limited to, neo (Potrykus et al., 1985), bar (Hinchee et al., 1988), bxn (Stalker et al., 1988); a mutant acetolactate synthase (ALS) (European Patent Application 154, 204, 1985) a methotrexate resistant DHFR (Thillet et al., 1988), β-glucuronidase (GUS); R-locus (Dellaporta et al., 1988), β-lactamase (Sutcliffe, 1978), xylE (Zukowsky et al., 1983), α-amylase (Ikuta et al., 1990), tyrosinase (Katz et al., 1983), β-galactosidase, luciferase (lux) (Ow et al., 1986), aequorin (Prasher et al., 1985), and green fluorescent protein (Sheen et al., 1995; Haseloff et al., 1997; Reichel et al., 1996; Tian et al., 1997; WO 97/41228).

Included within the terms “selectable” or “screenable” markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extension or tobacco PR-S).

### II. ANTISENSE AND RNAI CONSTRUCTS

In the methods and compositions of the present invention, NPR1 activity can be down-regulated by any means known in the art, including through the use of ribozymes or aptamers. NPR1 activity can also be down-regulated with an antisense or RNAi molecule.

In particular, constructs comprising an NPR1 coding sequence, including fragments thereof, in antisense orientation, or combinations of sense and antisense orientation, may be used to decrease or effectively eliminate the expression of the gene in a plant such as a citrus tree or variety. Accordingly, this may be used to “knock-out” the function of the coding sequence or homologous sequences thereof.

Techniques for RNAi are well known in the art and are described in, for example, Lehner et al., (2004) and Downward (2004). The technique is based on the ability of double stranded RNA to direct the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the invention, such an oligonucleotide may comprise any unique portion of a nucleic acid sequence provided herein. In certain embodiments of the invention, such a sequence comprises at least 18, 30, 50, 75, or 100 or more contiguous nucleic acids of the nucleic acid sequence of a lignin biosynthesis gene, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that an embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., as in a ribozyme) could be designed. Methods for selection and design of sequences that generate RNAi are well known in the art (e.g. Reynolds, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### III. METHODS FOR GENETIC TRANSFORMATION

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force.

### IV. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce, into the host cell, a marker gene which confers resistance to some normally inhibitory agent, such as an antibiotic or herbicide. Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

Cells that survive the exposure to the selective agent, or cells that have been scored positive in a screening assay, may be cultured in media that supports regeneration of plants. The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue.

To confirm the presence of the exogenous DNA in the regenerating plants, a variety of assays may be performed. Such assays include, for example, “molecular biological” assays, such as Southern and northern blotting and polymerase chain reaction (PCR); “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant. DNA integration into the host genome and the independent identities of transformants may be determined using, e.g., Southern hybridization or PCR. Expression may then be evaluated by specifically identifying the protein products of the introduced genes or evaluating the phenotypic changes brought about by their expression.

### V. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a selected lignin biosynthesis coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants.

As used herein the term “progeny” denotes the offspring of any generation of a parent plant prepared in accordance with the instant invention, wherein the progeny comprises a selected DNA construct. “Crossing” a plant to provide a plant line having one or more added transgenes relative to a starting plant line, as disclosed herein, is defined as the techniques that result in an NPR1 coding sequence of the invention being introduced into a plant line by crossing a starting line with a donor plant line that comprises a first selected DNA of the invention. To achieve this in a plant such as a citrus tree one could, for example, perform the following steps:

(a) plant seeds of the first (starting line) and second (donor plant line that comprises a first selected DNA of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

In some embodiments, asexual reproduction or propagation may be used to obtain a progeny plant in accordance with the invention. Techniques to achieve asexual propagation or reproduction in citrus trees or varieties may include, for example, grafting, budding, top-working, layering, runner division, cuttings, rooting, T-budding, and the like. In some embodiments, one citrus variety into which an NPR1 coding sequence has been introduced may be grafted onto the rootstock of another variety. In other embodiments, an NPR1 coding sequence may be introduced into the rootstock. In either of these situations, one or both of the plant varieties may exhibit increased tolerance or resistance to HLB.

### VI. DEFINITIONS

Expression: The combination of intracellular processes, including transcription and translation undergone by a coding DNA molecule such as a structural gene to produce a polypeptide or functional nucleic acid (e.g., an RNAi, antisense molecule, ribozyme, aptamer, etc.).

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be from another species, organism, plant, tree, or variety, or may be native to the host genome, but be rearranged with respect to other genetic sequences within the host sequence. For example, a regulatory sequence may be heterologous in that it is linked to a different coding sequence relative to the native regulatory sequence. In addition, a particular recombinant DNA molecule may be heterologous with respect to a cell or organism into which it is inserted when it would not naturally occur in that particular cell or organism

Overexpress: As used herein, “overexpress” refers to increased expression of a gene or coding sequence over that found in nature or a control plant or tissue. In some embodiments, “overexpress” may refer to greater expression of a gene or coding sequence in a transgenic plant into which a heterologous coding sequence is introduced, when compared to a plant lacking the heterologous coding sequence.

Plant: As used herein, a “plant” or “citrus plant” or “tree” refers to a citrus tree into which an NPR1 coding sequence has been introduced. Such a plant or tree exhibits increased tolerance or resistance to Huanglongbing (HLB) in accordance with the invention.

Plant part: As used herein, a “plant part” may include, but is not limited to a fruit, seed, tissue, stem, root, phloem, or flower of a citrus tree or variety described herein. A plant part may be any part of the plant from which another plant may arise.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus, or explant).

Rootstock: As used herein, a “rootstock” refers to underground plant parts such as roots, from which new above-ground growth of a plant or tree can be produced. In accordance with the invention, a rootstock may be used to grow a different variety through asexual propagation or reproduction such as grafting. As used herein, a “scion” refers to a plant part that is grafted onto a rootstock variety. A scion may be from the same or a different plant type or variety.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Development of Plant Transformation Vectors

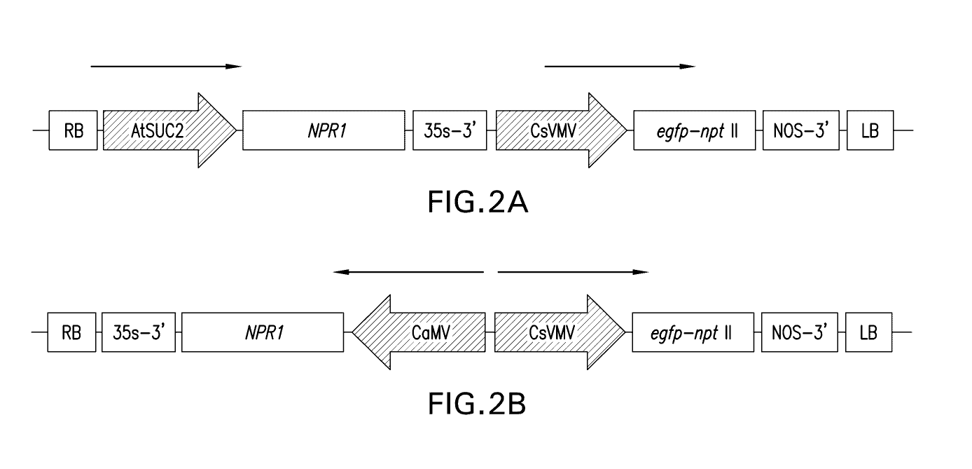

The cDNA sequence of AtNPR1 (U76707) is available in the NCBI database and provided as SEQ ID NO:20. Primers to amplify the AtNPR1 were designed using the bioinformatics software Vector NTI® (Life Technologies, NY, USA) to incorporate a BamHI restriction site at the 5′ end and a NotI site at the 3′ end. Total RNA was isolated from 100 mg of tissue from young, fully expanded leaves of Arabidopsis thaliana cv. Columbia using a RNeasy Mini Kit (Qiagen Inc., Valencia, Calif.). cDNA was synthesized from 500 ng total RNA using Oligo (dT) primer and a RETROscript® RT-PCR kit as described by the manufacturer (Applied Biosystems, Austin, Tex.). The cDNA product was used as a template for PCR using primers as described above. The gene was excised as a BamHI/NotI fragment and inserted between a double enhanced CaMV 35S promoter (d35S) and a CaMV 35S terminator (3′CaMV) of a pUC18-derived plasmid pDR. Variations of this cloning vector containing the phloem-specific Arabidopsis SUC2 promoter (NCBI accession: JQ733913) were also produced. A HindIII DNA fragment containing the expression cassette d35S (or AtSUC2)—NPR1 gene—3′CaMV were isolated and cloned into the unique HindIII site of a pBIN19-derived binary vector. This vector, containing a bifunctional nptII/egfp fusion gene has been described earlier [21]. All constructions were verified first by restriction analysis and then by DNA sequencing (FIG. 2). Each vector was introduced into A. tumefaciens strain EHA105 [22] by the freeze-thaw method [23].

### Example 2

### Transformation, Selection and Propagation of Regenerants

Agrobacterium mediated transformation of etiolated sweet orange epicotyl segments from the cultivars ‘Hamlin’ and ‘Valencia’ were carried out as described previously [24]. EGFP-specific fluorescence in putative transgenic lines was evaluated using a Zeiss SV11 epi-fluorescence stereomicroscope equipped with a light source consisting of a 100 W mercury bulb and a FITC/GFP filter set with a 480 nm excitation filter and a 515 nm longpass emission filter producing a blue light (Chroma Technology Corp., VT, USA). Transgenic ‘Hamlin’ and ‘Valencia’ sweet orange shoots are very difficult to root in vitro [24] and in this study no attempt was made to root any of the EGFP expressing transgenic lines. Instead, EGFP positive transgenic shoots were micrografted in vitro onto 1 month old Carrizo citrange (Citrus sinensis (L.) Osbeck× Poncirus trifoliata (L.) Raf.) nucellar rootstock seedlings. After a month of growth in vitro, the grafted shoots were potted into a peat based commercial potting medium (Metromix 500, Sun Gro Horticulture, Bellevue, Wash.) and acclimated under greenhouse conditions. An ex vitro micrografting technique was subsequently used to rapidly propagate transgenic plants onto 6 month old Carrizo rootstocks [25]. Plants were grown for an additional 9 to 12 months before evaluation of disease resistance. Transgenic lines with the AtSUC2-NPR1 construct had an ‘A’ added in as a suffix.

### Example 3

### Molecular Analysis of Transformants

Citrus genomic DNA, was isolated from 100 mg of young transgenic leaf tissues using the GenElute™ Plant Genomic DNA Miniprep Kit (Sigma-Aldrich Corp., St. Louis, Mo.). Duplex PCR was carried out in a thermal cycler (MJ Research, Watertown, Mass.) using GoTaq® Green Master PCR Mix (Promega Corp, Madison Wis.) and appropriate primers NP51, 5′ ATG GAC ACC ACC ATT GAT GGA TTC 3′ (SEQ ID NO:1) and NP32, 5′ ACG ACG ATG AGA GAG TTT ACG GTT AG 3′ (SEQ ID NO:2), and EG-51, 5′ATG GTG AGC AAG GGC GAG GAG CTG T3′ (SEQ ID NO:3) and EG-32, 5′CTT GTA CAG CTC GTC CAT GCC GAG A3′ (SEQ ID NO:4) to confirm the presence of the AtNPR1 and egfp transgenes respectively in the genome of transgenic citrus plants. Amplified DNA fragments were electrophoresed on a 1% agarose gel and visualized under UV light. All images were recorded and digitized. All samples for the detection of Clas in transgenic citrus were analyzed by qPCR at the diagnostic laboratory of Southern Gardens Citrus in Clewiston, Fla., USA. Four to five fully expanded and hardened leaves were collected from dark green colored branches.

Leaves were placed in a zip lock plastic bag, kept cool and out of direct sunlight and subsequently shipped by overnight mail and processed the following day. DNA was isolated from 100 mg of leaf petiole tissue using BioSprint DNA Plant kits (Qiagen, Valencia, Calif.) on a BioSprint 96 instrument (Qiagen, Valencia, Calif.). DNA was dissolved in 100 μl of TE buffer and 2 μl were used for qPCR. qPCR was performed in a 25 μl volume on an AB17300 (Life Technologies, Grand Island, N.Y.) using TaqMan Universal PCR Master Mix (Life Technologies, Grand Island, N.Y.) using the Li primers [26].

Southern blot analysis was carried out for confirmation of copy number in selected transgenic citrus plants. Fifteen μg of EcoRI digested genomic DNA immobilized on a positively-charged nylon membrane was probed with a DIG-labeled AtNPR1 probe. Following hybridization to the probe, the chemiluminescence substrate CDP-Star was used for immunological detection of hybridization signals using X-ray film autography. Validation of transgene copy number was carried out using qPCR essentially as described previously [27].

RNA was isolated from 100 mg of leaf tissue using an RNeasy Mini Kit (Qiagen Inc. Valencia, Calif.) according to the manufacturer's protocol. Real-time quantitative PCR (RT-qPCR) was performed as outlined before with minor modifications [28]. The RT-qPCR reactions were performed with a final volume of 25 μl using the TaqMan® RNA-to-Ct™ one-step kit (Applied Biosystems, Foster City, Calif.) according to the manufacturer's instructions. The one-step kit parameters consisted of 20 min incubation at 48° C. followed by 10 min incubation at 95° C. and 40 cycles at 95° C. for 15 s and 60° C. for 1 min. Each qPCR contained negative and non-template/water controls in addition to the sample being tested. Experiments were repeated at least twice with three replicates and the data was analyzed using Applied Biosystems software version 2.0.6. Relative quantitation was measured using the comparative Cq method (2-ΔΔCt). The fold change in the relative expression was then determined by calculating 2-ΔΔCt [29]. The sequences of the primers and probes including the reporter fluorescent dye and dark quencher dye used in the RT-qPCR are shown in Table 1. The 18S rRNA housekeeping gene was used as an endogenous control. A set of reaction mixtures composed of one to five copies of AtNPR1 gene equivalences, was used to establish a standard curve for the evaluation of transgene copy number. The method as outlined earlier was used to prepare the set of reaction mixtures [30].

TABLE 1
Primers used in real-time PCR assay of transgenic citrus plants.

| SEQ | | Amplicon | |
|---|---|---|---|
| ID NO | Target gene | length (bp) | Primer/probe sequence 5′→3′^{A} |
| 5 | AtNPR1 | 113 bp | TGCATCAGAAGCAACTTTGG |
| 6 | | | 6FAM-CGCAAAACAAGCCACTATG |
| 7 | | | GGCCTTTGAGAGAATGCTTG |
| 8 | CsPR1 | 88 bp | AACTCGCCTCAAGACTACCT |
| 9 | | | 6FAM-TCACAATTCAGCTCGAGCAGCAGTC |
| 10 | | | TGCAACTGTGTCGTTCCATA |
| 11 | CsPR2 | 92 bp | ACTTCGCTCAGTACCTTGTTC |
| 12 | | | 6FAM-ATCAACAGAGCCGGCCTTGGAAA |
| 13 | | | GGCAGTGGAAACCTTGATTTG |
| 14 | CsWRKY70 | 106 bp | CTGTGCTCGGTACTACTGTTAC |
| 15 | | | 6FAM-TGAGAAGTATCAGCAGCAGCAGGC |
| 16 | | | CGGCGATAGTCATCGGAATTA |
| 17 | 18S rRNA | 112 bp | TCGGGTGTTTTCACGTCTCA |
| 18 | | | HEX-TGGAACTCTTGGATTTTGCC |
| 19 | | | CGCCGTAGGTGAGGTAGC |

^{A}Primer/probe sequences listed in the column include the forward primer in
the first line, followed by the probe sequence in the second and the reverse
primer sequence in the third line.

### Example 4

### HLB Resistance Studies

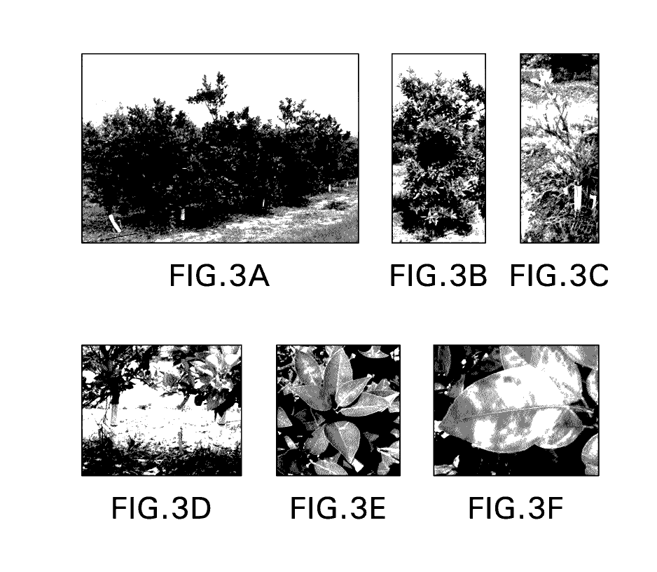

Disease resistance to HLB in this study was evaluated in two ways. First, in a no-choice greenhouse evaluation study, 3 replicated clones of independent transgenic plant lines were exposed to free flying CLas positive ACP continuously for two years. Trees were routinely pruned and fertilized with both 9 month slow release and liquid fertilizer to stimulate new flush production. These trees were evaluated every 6 months for two years for the presence of HLB by qPCR as outlined previously. ACP were also randomly evaluated during this study for the presence of the CLas. In the second concurrent study, selected transgenic trees and controls (consisting of 10% of the total tree population) were planted in a high disease pressure (over 90% infection rate) field site in a randomized block design experiment. In the test site, trees were planted at a narrow spacing of 2 feet to maximize land utilization (FIG. 3D). These trees were similarly evaluated every 6 months for three years by qPCR for the presence of HLB. Data were analyzed to calculate standard error using MS Excel.

### Example 5

### Production of Genetically Modified Citrus Plants

A total of 36 transgenic ‘Hamlin’ and ‘Valencia’ sweet orange lines expressing the 35S-NPR1 construct and 22 lines with the AtSUC2-NPR1 construct were regenerated. Transgenic plants were selected based on visual selection using EGFP fluorescence. A 20% transformation efficiency was observed using ‘Hamlin’ epicotyl segments while the transformation efficiency using ‘Valencia’ epicotyl segments were significantly lower (3%). These shoots were micrografted onto Carrizo seedlings to expedite plant growth. In vitro micro grafted shoots were hardened after 4-6 weeks of grafting and transferred to a greenhouse. After 4 months of growth, plants were analyzed for the presence of the AtNPR1 gene before being micro grafted ex vitro [25]. Major phenotypic abnormalities were not observed in a majority of the transgenic plants regenerated in this study, although 2 lines obtained with the 35S-NPR1 construct exhibited abnormally slow growth and were deemed unsuitable and discarded.

### Example 6

### Verification of Transgene Integration, Transcript Accumulation, and Transgene Copy

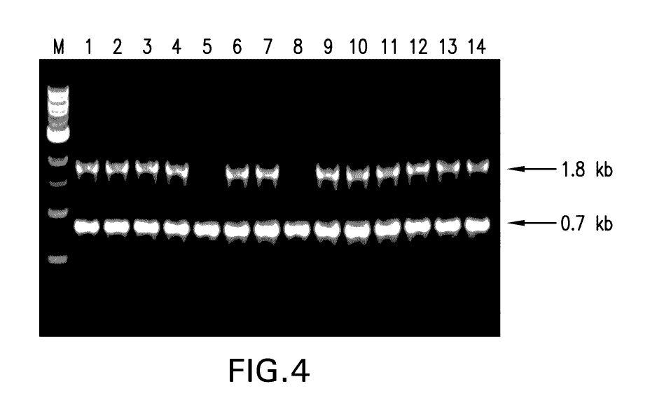

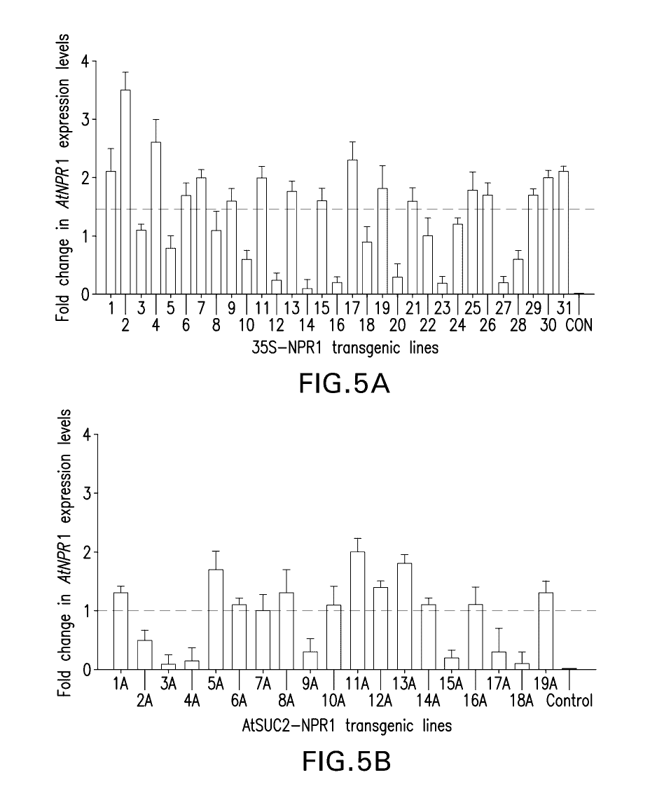

Thirty one transgenic Hamlin′ and ‘Valencia’ sweet orange lines expressing the 35S-NPR1 construct had both the egfp as well as the AtNPR1 genes incorporated into the genome as confirmed by PCR. Nineteen lines with the AtSUC2-NPR1 construct behaved similarly Results from 14 arbitrarily selected samples are shown in FIG. 4. Lines without the AtNPR1 gene were discarded and the rest analyzed for mRNA production through qRT-PCR (FIG. 5). Transgenic lines with the 35S-NPR1 construct that had a 1.5-fold higher level of expression were considered to exhibit a relative high level of expression of AtNPR1. In a similar manner, transgenic lines with the weaker phloem specific AtSUC2-NPR1 construct that had a 1-fold higher level of expression were considered to exhibit a relative high level of expression of AtNPR1. Seventeen constitutively expressing lines (35S-NPR1) and 12 phloem specific lines (AtSUC2-NPR1) that were considered to have a relative high level of expression of AtNPR1 were obtained (FIG. 5; dotted line). The transgenic lines were subsequently micrografted ex vitro to produce a population of trees for disease resistance analyses. Based on the greenhouse and field results, transgenic lines could be categorized as asymptomatic and resistant, symptomatic but tolerant or susceptible to HLB. Lines 2, 4, 9A, 11A and 18 were selected as representatives of the transgenic population for detailed molecular analyses.

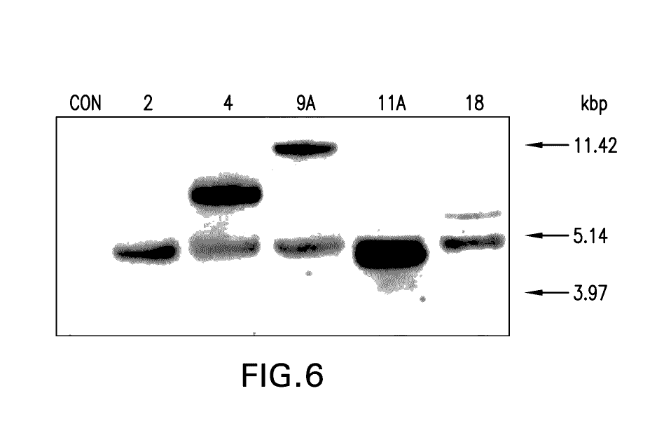

Southern blot hybridization was used to determine the number of inserted AtNPR1 copies in the genomes of the selected transgenic lines. All transgenic lines demonstrated AtNPR1 integration profiles, whereas none was detected from the control plant (FIG. 6). Transgenic line 2 had one gene copy integrated into the genome. Transgenic lines 2, 4, 9A, and 11A are ‘Hamlin’, while line 18 is a ‘Valencia’ sweet orange. Transgenic plant lines 4, 9A, and 18 had 2 copies, while based on the intensity of the band it was predicted that 11A line had 2 to 3 copies. These results were confirmed by q-PCR (Table 2). No amplification was detected from a non-transformed control plant.

TABLE 2
Transgene copy number determination using quantitative
real-time PCR by comparison of transgenic lines
with external plasmid controls.

| Transgenic Line | Cultivar | Mean [symbol] ^{A} | STD [symbol] ^{B} | Mean Conc. [symbol] |
|---|---|---|---|---|
| NPR1-2 | Hamlin | 25.550 | 0.115 | 1.101 |
| NPR1-4 | Hamlin | 25.160 | 1.378 | 1.605 |
| NPR1-9A | Hamlin | 25.064 | 0.639 | 1.738 |
| NPR1-11A | Hamlin | 24.412 | 0.867 | 2.643 |
| NPR1-18 | Valencia | 24.738 | 0.184 | 2.190 |
| Plasmid-1C^{E} | — | 25.727 | 0.132 | 0.818 |
| Plasmid-2C | — | 24.746 | 0.079 | 2.180 |
| Plasmid-3C | — | 24.022 | 0.149 | 3.185 |
| Plasmid-4C | — | 23.567 | 0.115 | 3.816 |
| Plasmid-5C | — | 23.107 | 0.232 | 4.753 |

^{A}Average values of crossing point from three sample replicates.
^{B}Standard deviations.
^{C}Average values of extrapolated concentration relative to a single transgene copy.
^{D}Copy number.
^{E}Plasmid DNA used for copy number calculations.

### Example 7

### PR Gene Expression

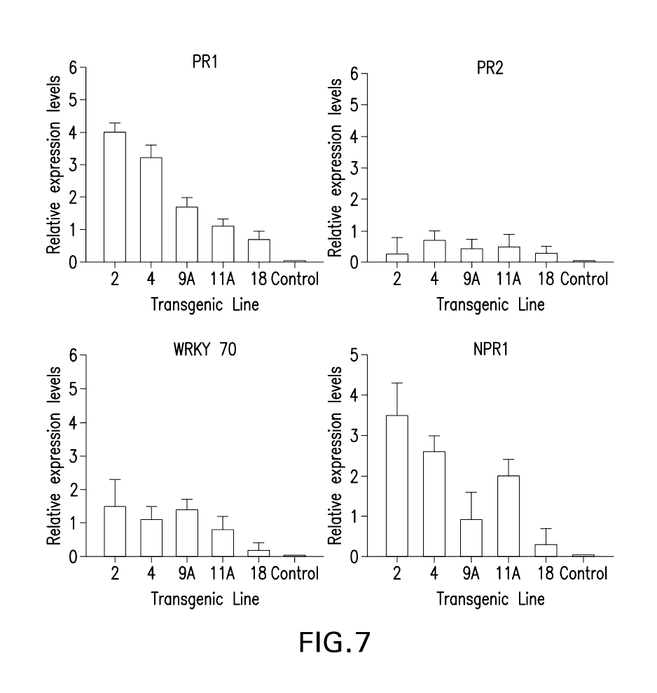

The selected transgenic lines that were analyzed for copy number by Southern blot hybridization were also evaluated for PR gene expression using qRT-PCR. The pathogenesis-related PR1 gene is induced by NPR1 [31] and significant variation in PR1 gene expression was observed in these transgenic lines (FIG. 7). All tested lines had enhanced PR1 gene expression. Transgenic line 2 exhibited a four-fold level in PR1 gene expression compared to the control. Lower expression levels were observed in the other lines. Expression levels of the PR2 gene, a SAR marker gene in citrusm were also higher in all transgenic lines evaluated. However, gene expression was less than 1-fold higher in all lines analyzed and there was no statistical significance between the evaluated transgenic lines. WRKY70 is a direct target for NPR1 and plays a role as a positive regulator of SA-mediated gene expression and resistance [32]. WRKY70 expression was not observed to be significantly different in any NPR1 overexpressing transgenic line except in line 18. The AtNPR1 expression levels in the transgenic lines were many fold higher than that observed in the non-transformed control plant, except for transgenic line 11A (FIGS. 5 and 7).

### Example 8

### Susceptibility of Transformed Lines to Huanglongbing

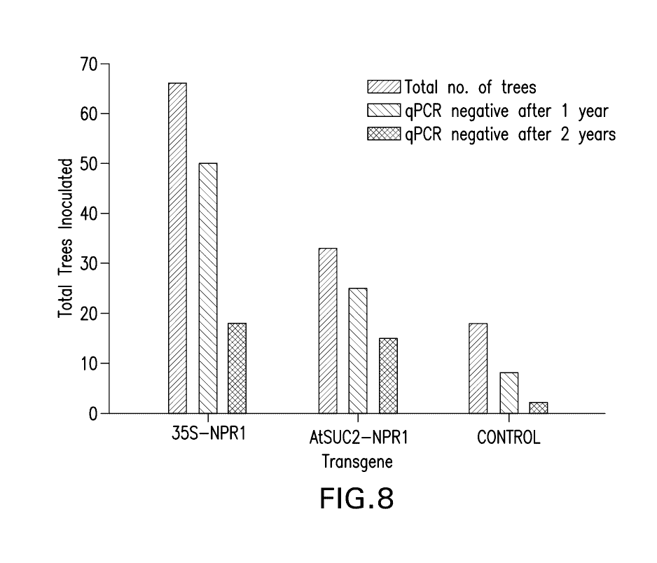

Huanglongbing (HLB) is caused by the phloem-limited, fastidious α-proteobacteria CLas spp. A majority of the trees tested positive for the bacterium in the second year of evaluation. Approximately 45% of the trees expressing AtNPR1 under the control of the phloem specific promoter were HLB negative, while 27% of the trees expressing AtNPR1 under the control of the constitutive 35S promoter remained HLB negative (FIG. 8). The bacterium was not detected in transgenic lines 2, 4, and 9A for the duration of this study. Transgenic line 11A tested positive within 6 months, and the severely infected trees were discarded after 18 months of infection (Table 3). Control trees tested positive for the presence of the CLas within 6 months after infection and remained positive for the entire duration of the study. In the second study, trees were planted in a high disease pressure field site. The results from that study are presented in Table 4. Transgenic line 2 remained CLas free for the duration of the study except for the 24-month sampling period when it tested positive. Line 4 tested positive at the 30-month sampling period, while line 9A tested positive at 30 months but was CLas free at 36 months. Both of these lines did not decline in health and showed continued growth with periodic flushes. Line 11A tested positive after 18 months in the field and remained CLas positive for the duration of the test period. The tolerant lines 2 and 9A also did not demonstrate any visual symptoms for the duration of the study, while line 4 developed symptoms, tested positive for CLas but continued growth at a similar rate to lines 2 and 9A. Transgenic line 18 was the most susceptible line and tested positive for CLas within 6 months after planting. Trees from this line began dying after 30 months in the field and were all dead within 36 months of planting in the field. Similar results were observed in the non-transgenic control trees (FIG. 3).

TABLE 3
Quantification of CLas bacterial titers following qPCR from leaf petiole
and midribs of the transgenic plants and controls grown under greenhouse
conditions and exposed to free flying, potentially CLas-positive psyllids. The mean
threshold cycle values (Ct) at specified time intervals are demonstrated.

| | Transgenic | Transgenic | Transgenic | Transgenic | Transgenic | |
|---|---|---|---|---|---|---|
| | line #2 | line #4 | line #9A | line #11A | line #18 | Control |
| 6 months | — | — | — | 29.29 ± 2.3 | NT | 23.48 ± 1 |
| 12 months | — | — | — | 23.63 ± 1.4 | NT | 22.04 ± 2.2 |
| 18 months | — | — | — | 21.19 ± 3.1 | NT | 20.78 ± 4.5 |
| 24 months | — | — | — | * | NT | * |

* dead trees,
NT; not tested, Standard errors were calculated from three replicates

TABLE 4
Quantification of CLas bacterial titers following qPCR from leaf petiole
and midribs of the transgenic plants and controls grown under field conditions in a
high disease pressure test site. The mean threshold cycle values (Ct) at specified time
intervals are demonstrated.

| | Transgenic | Transgenic | Transgenic | Transgenic | Transgenic | |
|---|---|---|---|---|---|---|
| | line #2 | line #4 | line #9A | line #11A | line #18 | Control |
| 6 months | — | — | — | — | 37.42 ± 3.1 | 38.39 ± 3.3 |
| 12 months | — | — | — | — | 30.13 ± 2.5 | 26.20 ± 1.8 |
| 18 months | — | — | — | 33.81 ± 4.1 | 23.81 ± 4.1 | 27.69 ± 1.5 |
| 24 months | 36.00^{a} | — | — | 27.72 ± 2.3 | 29.02 ± 1.6 | 22.87 ± 1.6 |
| 30 months | — | 33.02 ± 2.4 | 38.98^{a} | 24.45 ± 2.2 | 21.52 ± 3.1 | 23.14 ± 2.3 |
| 36 Months | — | 29.69 ± 5.1 | — | 26.16 ± 4.6 | * | * |

* dead trees,
^{a}only one replicate was PCR-positive.
Standard errors were calculated from three replicates.

In addition to inducing resistance to HLB, the SAR response observed could potentially protect citrus trees from other important citrus fungal and bacterial diseases such as citrus canker and black spot. Both constitutive expression and phloem expression of AtNPR1 would lead to a genetically modified commercial scion and in addition, phloem expression could lead to the development of a transposable transgene effect that could possibly induce HLB resistance in non-transgenic citrus. Phloem specific expression of the transgene and the observed resistance could allow the movement of the SAR response across the graft union. This transfer may induce a SAR response that could potentially protect the non-transgenic scion from HLB. In this model any existing non-transgenic scion could be budded onto a transgenic rootstock in order to impart HLB resistance. A non-transgenic scion grafted onto a transgenic rootstock could potentially be acceptable to the consumer than transgenic citrus scions. In addition, this transgene can also serve as a component for the development of an all plant T-DNA derived consumer friendly GM tree.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Duan Y, Zhou L, Hall D G, Li W, Doddapaneni H, Lin H, et al. Complete genome sequence of citrus huanglongbing bacterium, ‘Candidatus Liberibacter asiaticus’ obtained through metagenomics. Molecular plant-microbe interactions: MPMI. 2009; 22(8):1011-20. Epub 2009/07/11. doi: 10.1094/mpmi-22-8-1011. PubMed PMID: 19589076.
2. Alves G R, Diniz A J, Parra J R. Biology of the Huanglongbing vector Diaphorina citri (Hemiptera: Liviidae) on different host plants. Journal of economic entomology. 2014; 107(2):691-6. Epub 2014/04/30. PubMed PMID: 24772551.
3. Manjunath K L, Halbert S E, Ramadugu C, Webb S, Lee R F. Detection of ‘Candidatus Liberibacter asiaticus’ in Diaphorina citri and Its Importance in the Management of Citrus Huanglongbing in Florida. Phytopathology. 2008; 98(4):387-96. doi: 10.1094/PHYTO-98-4-0387.
4. Wang N, Trivedi P. Citrus Huanglongbing: A Newly Relevant Disease Presents Unprecedented Challenges. Phytopathology. 2013; 103(7):652-65. doi: 10.1094/PHYTO-12-12-0331-RVW.
5. Massenti R, Lo Bianco R, Sandhu A K, Liwei G, Sims C. Huanglongbing modifies quality components and flavonoid content of ‘Valencia’ oranges. Journal of the science of food and agriculture. 2014. Epub 2014/12/30. doi: 10.1002/jsfa.7061. PubMed PMID: 25546309.
6. Qureshi J A, Kostyk B C, Stansly P A. Insecticidal Suppression of Asian Citrus Psyllid Diaphorina citri (Hemiptera: Liviidae) Vector of Huanglongbing Pathogens. PLoS ONE. 2014; 9(12):e112331. doi: 10.1371/journal.pone.0112331. PubMed PMID: PMC4249845.
7. Belasque Júnior J, Yamamoto P T, Miranda M Pd, Bassanezi R B, Ayres A J, Bové J M. Huanglongbing control in São Paulo State, Brazil. Citrus Research and Technology. 2010; 31(1):53-64. doi: 10.5935/2236-3122.20100005.
8. Westbrook C J, Hall D G, Stover E, Duan Y P, Lee R F. Colonization of Citrus and citrus-related germplasm by Diaphorina citri (Hemiptera: Psyllidae). HortScience. 2011; 46(7):997-1005.
9. Furr J, Cooper W, Reece P. An investigation of flower formation in adult and juvenile citrus trees. American Journal of Botany. 1947:1-8.
10. Gmitter J, F. G., Grosser J W, Castle W S, Moore G A. A comprehensive Citrus genetic improvement program. In: Khan I A, editor. Citrus Genetics, Breeding and Biotechnology. Oxfordshire, U K: CAB International; 2007. p. 9-18.
11. Zhang X, Francis M I, Dawson W O, Graham J H, Orbovie V, Triplett E W, et al. Over-expression of the Arabidopsis NPR1 gene in citrus increases resistance to citrus canker. Eur J Plant Pathol. 2010; 128(1):91-100.
12. Kuć J. Induced Immunity to Plant Disease. BioScience. 1982; 32(11):854-60. doi: 10.2307/1309008.
13. Ward E R, Uknes S J, Williams S C, Dincher S S, Wiederhold D L, Alexander D C, et al. Coordinate Gene Activity in Response to Agents That Induce Systemic Acquired Resistance. The Plant Cell. 1991; 3(10):1085-94. PubMed PMID: PMC160074.
14. Malamy J, Carr J P, Klessig D F, Raskin I. Salicylic Acid: a likely endogenous signal in the resistance response of tobacco to viral infection. Science (New York, N.Y.). 1990; 250(4983):1002-4. Epub 1990/11/16. doi: 10.1126/science.250.4983.1002. PubMed PMID: 17746925.
15. Durrant W E, Dong X. Systemic acquired resistance. Annual review of phytopathology. 2004; 42:185-209. Epub 2004/07/31. doi:10.1146/annurev.phyto.42.040803.140421. PubMed PMID: 15283665.
16. Forouhar F, Yang Y, Kumar D, Chen Y, Fridman E, Park S W, et al. Structural and biochemical studies identify tobacco SABP2 as a methyl salicylate esterase and implicate it in plant innate immunity. Proceedings of the National Academy of Sciences of the United States of America. 2005; 102(5):1773-8. Epub 2005/01/26. doi: 10.1073/pnas.0409227102. PubMed PMID: 15668381; PubMed Central PMCID: PMCPmc547883.
17. Nawrath C, Metraux J P. Salicylic acid induction-deficient mutants of Arabidopsis express PR-2 and PR-5 and accumulate high levels of camalexin after pathogen inoculation. Plant Cell. 1999; 11(8):1393-404. Epub 1999/08/17. PubMed PMID: 10449575; PubMed Central PMCID: PMCPmc144293.
18. Kinkema M, Fan W, Dong X. Nuclear Localization of NPR1 Is Required for Activation of P R Gene Expression. The Plant Cell. 2000; 12(12):2339-50. doi: 10.1105/tpc.12.12.2339.
19. Clarke J D, Liu Y, Klessig D F, Dong X. Uncoupling P R gene expression from NPR1 and bacterial resistance: characterization of the dominant Arabidopsis cpr6-1 mutant. Plant Cell. 24 1998; 10(4):557-69. Epub 1998/06/13. PubMed PMID: 9548982; PubMed Central PMCID: 25 PMCPmc144011.
20. Cao H, Li X, Dong X. Generation of broad-spectrum disease resistance by overexpression of an essential regulatory gene in systemic acquired resistance. Proceedings of the National Academy of Sciences of the United States of America. 1998; 95(11):6531-6. Epub 1998/05/30. PubMed PMID: 9601001; PubMed Central PMCID: PMCPmc34547.
21. Li Z, Jayasankar S, Gray D J. Expression of a bifunctional green fluorescent protein (GFP) fusion marker under the control of three constitutive promoters and enhanced derivatives in transgenic grape (Vitis vinifera). Plant science: an international journal of experimental plant biology. 2001; 160(5):877-87. Epub 2001/04/12. PubMed PMID: 11297784.
22. Hood E, Gelvin S, Melchers L, Hoekema A. New Agrobacterium helper plasmids for gene transfer to plants. Transgenic Research. 1993; 2(4):208-18. doi: 10.1007/BF01977351.
23. Burow M, Chlan C, Sen P, Lisca A, Murai N. High-frequency generation of transgenic tobacco plants after modified leaf disk cocultivation withAgrobacterium tumefaciens. Plant Mol Biol Rep. 1990; 8(2):124-39. doi: 10.1007/BF02669766.
24. Dutt M, Grosser J W. Evaluation of parameters affecting Agrobacterium-mediated transformation of citrus. Plant Cell Tiss Organ Cult. 2009; 98(3):331-40. doi: 10.1007/s11240-009-9567-1.
25. Dutt M, Grosser J. An embryogenic suspension cell culture system for Agrobacterium-mediated transformation of citrus. Plant cell reports. 2010; 29(11):1251-60.
26. Li W, Hartung J S, Levy L. Quantitative real-time PCR for detection and identification of Candidatus Liberibacter species associated with citrus huanglongbing. Journal of microbiological methods. 2006; 66(1):104-15.
27. Dutt M, Li Z, Dhekney S, Gray D. A co-transformation system to produce transgenic grapevines free of marker genes. Plant Science. 2008; 175(3):423-30.
28. Dutt M, Ananthakrishnan G, Jaromin M, Brlansky R, Grosser J. Evaluation of four phloem-specific promoters in vegetative tissues of transgenic citrus plants. Tree physiology. 2012; 32(1):83-93.
29. Livak K J, Schmittgen T D. Analysis of Relative Gene Expression Data Using Real-Time Quantitative PCR and the 2AACT Method. methods. 2001; 25(4):402-8.
30. Song P, Cal C, Skokut M, Kosegi B, Petolino J. Quantitative real-time PCR as a screening tool for estimating transgene copy number in WHISKERS™-derived transgenic maize. Plant Cell Reports. 2002; 20(10):948-54. doi: 10.1007/s00299-001-0432-x.
31. Zhou J-M, Trifa Y, Silva H, Pontier D, Lam E, Shah J, et al. NPR1 Differentially Interacts with Members of the TGA/OBF Family of Transcription Factors That Bind an Element of the PR-1 Gene Required for Induction by Salicylic Acid. Molecular Plant-Microbe Interactions. 2000; 13(2):191-202. doi: 10.1094/MPMI.2000.13.2.191.
32. Wang D, Amornsiripanitch N, Dong X. A genomic approach to identify regulatory nodes in the transcriptional network of systemic acquired resistance in plants. PLoS pathogens. 2006; 2(11):e123.
34. Morris A, Muraro R P, Castle W S. Optimal grove replanting to mitigate endemic HLB. Citrus Ind. 2011; 92(4):12-6.
35. Kim J S, Sagaram U S, Burns J K, Li J L, Wang N. Response of sweet orange (Citrus sinensis) to ‘Candidatus Liberibacter asiaticus’ infection: microscopy and microarray analyses. Phytopathology. 2009; 99(1):50-7. Epub 2008/12/06. doi: 10.1094/phyto-99-1-0050. PubMed PMID: 19055434.
36. Fang D, Roose M. Identification of closely related citrus cultivars with inter-simple sequence repeat markers. Theoretical and Applied Genetics. 1997; 95(3):408-17.
37. Zhang Y, Fan W, Kinkema M, Li X, Dong X. Interaction of NPR1 with basic leucine zipper protein transcription factors that bind sequences required for salicylic acid induction of the PR-1 gene. Proceedings of the National Academy of Sciences. 1999; 96(11):6523-8.
38. Cao H, Glazebrook J, Clarke J D, Volko S, Dong X. The Arabidopsis NPR1 gene that controls systemic acquired resistance encodes a novel protein containing ankyrin repeats. Cell. 1997; 88(1):57-63.
39. Makandar R, Essig J S, Schapaugh M A, Trick H N, Shah J. Genetically Engineered Resistance to Fusarium Head Blight in Wheat by Expression of Arabidopsis NPR1. Molecular Plant-Microbe Interactions. 2006; 19(2):123-9. doi: 10.1094/MPMI-19-0123.
40. Kumar V, Joshi S, Bell A, Rathore K. Enhanced resistance against Thielaviopsis basicola in transgenic cotton plants expressing Arabidopsis NPR1 gene. Transgenic Research. 2013; 22(2):359-68. doi: 10.1007/s11248-012-9652-9.
41. Silva K J P, Brunings A, Peres N A, Mou Z, Folta K M. The Arabidopsis NPR1 gene confers broad-spectrum disease resistance in strawberry. Transgenic research. 2015:1-12.
42. Lin W C, Lu C F, Wu J W, Cheng M L, Lin Y M, Yang N S, et al. Transgenic tomato plants expressing the Arabidopsis NPR1 gene display enhanced resistance to a spectrum of fungal and bacterial diseases. Transgenic Research. 2004; 13(6):567-81.
43. Wally O, Jayaraj J, Punja Z K. Broad-spectrum disease resistance to necrotrophic and biotrophic pathogens in transgenic carrots (Daucus carota L.) expressing an Arabidopsis NPR1 gene. Planta. 2009; 231(1): 131-41.
44. Chen X, Barnaby J Y, Sreedharan A, Huang X, Orbovie V, Grosser J W, et al. Over-expression of the citrus gene CtNH1 confers resistance to bacterial canker disease. Physiological and Molecular Plant Pathology. 2013; 84:115-22.
45. Zhong X, Xi L, Lian Q, Luo X, Wu Z, Seng S, et al. The NPR1 homolog GhNPR1 plays an important role in the defense response of Gladiolus hybridus. Plant Cell Rep. 2015; 34(6):1063-74. Epub 2015/02/25. doi: 10.1007/s00299-015-1765-1. PubMed PMID: 25708873.
46. Le Henanff G, Heitz T, Mestre P, Mutterer J, Walter B, Chong J. Characterization of Vitis vinifera NPR1 homologs involved in the regulation of pathogenesis-related gene expression. BMC Plant Biology. 2009; 9(1):54.
47. Chern M, Fitzgerald H A, Canlas P E, Navarre D A, Ronald P C. Overexpression of a rice NPR1 homolog leads to constitutive activation of defense response and hypersensitivity to light. Mol Plant Microbe Interact. 2005; 18(6):511-20. Epub 2005/07/01. doi: 10.1094/mpmi-18-0511. PubMed PMID: 15986920.
48. Chen J C, Lu H C, Chen C E, Hsu H F, Chen H H, Yeh H H. The NPR1 ortholog PhaNPR1 is required for the induction of PhaPR1 in Phalaenopsis aphrodite. Botanical Studies. 2013; 54(1):1-11.
49. Kuykendall L D, Murphy T S, Shao J, McGrath J M. Nucleotide sequence analyses of a sugarbeet genomic NPR1-class disease resistance gene. Journal of Sugar Beet Research. 2007; 44(1/2):35.
50. Meyer P, Saedler H. Homology-dependent gene silencing in plants. Annual review of plant biology. 1996; 47(1):23-48.
51. Kooter J M, Matzke M A, Meyer P. Listening to the silent genes: transgene silencing, gene regulation and pathogen control. Trends in plant science. 1999; 4(9):340-7.
52. Dutt M, Vasconcellos M, Grosser J. Effects of antioxidants on Agrobacterium-mediated transformation and accelerated production of transgenic plants of Mexican lime (Citrus aurantifolia Swingle). Plant Cell, Tissue and Organ Culture (PCTOC). 2011; 107(1):79-89.
53. Mou Z, Fan W, Dong X. Inducers of plant systemic acquired resistance regulate NPR1 function through redox changes. Cell. 2003; 113(7):935-44.
54. Maleck K, Dietrich R A. Defense on multiple fronts: how do plants cope with diverse enemies? Trends in plant science. 1999; 4(6):215-9.
55. Yu D, Chen C, Chen Z. Evidence for an Important Role of WRKY DNA Binding Proteins in the Regulation of NPR1 Gene Expression. The Plant Cell. 2001; 13(7):1527-40. doi: 10.1105/tpc.010115.
56. Li J, Brader G, Palva E T. The WRKY70 transcription factor: a node of convergence for jasmonate-mediated and salicylate-mediated signals in plant defense. The Plant Cell Online. 2004; 16(2):319-31.
57. Llorens E, Vicedo B, López M M, Lapeña L, Graham J H, Garcíα-Agustín P. Induced resistance in sweet orange against Xanthomonas citri subsp. citri by hexanoic acid. Crop Protection. 2015; 74:77-84.
58. Ülker B, Mukhtar M S, Somssich I E. The WRKY70 transcription factor of Arabidopsis influences both the plant senescence and defense signaling pathways. Planta. 2007; 226(1): 125-37.
59. Matzke A J, Matzke M A. Position effects and epigenetic silencing of plant transgenes. Current opinion in plant biology. 1998; 1(2):142-48.
60. Kooter J M, Matzke M A, Meyer P. Listening to the silent genes: transgene silencing, gene regulation and pathogen control. Trends in plant science. 1999; 4(9):340-47.

What is claimed is:

1. A citrus tree comprising an NPR1 coding sequence, wherein the citrus tree exhibits increased tolerance to Huanglongbing compared to a plant not comprising said NPR1 coding sequence.

2. The citrus tree of claim 1, wherein the NPR1 coding sequence is from Arabidopsis thaliana.

3. The citrus tree of claim 2, wherein the NPR1 coding sequence comprises a nucleic acid sequence encoding a polypeptide having at least 85% identity to the sequence of SEQ ID NO:21.

4. The citrus tree of claim 3, wherein the NPR1 coding sequence comprises a nucleic acid sequence having at least 85% identity to the sequence of SEQ ID NO:20.

5. The citrus tree of claim 1, wherein the tree overexpresses NPR1 in the phloem tissues.

6. The citrus tree of claim 1, wherein the citrus tree is a sweet orange tree.

7. The citrus tree of claim 1, wherein the sweet orange tree is a ‘Hamlin’ or a ‘Valencia’ sweet orange tree.

8. The citrus tree of claim 1, wherein the NPR1 coding sequence is operably linked to a heterologous promoter active in plant tissue.

9. The citrus tree of claim 8, wherein the heterologous promoter comprises a phloem-specific promoter or a constitutive promoter.

10. The citrus tree of claim 9, wherein the phloem-specific promoter comprises an Arabidopsis sucrose-proton symporter 2 (AtSUC2) promoter, or said constitutive promoter comprises a CaMV 35S promoter.

11. The citrus tree of claim 1 wherein said tolerance to Huanglongbing is the result of systemic acquired resistance (SAR).

12. A seed, cell, tissue, progeny, or part of the citrus tree of claim 1, wherein the seed, cell, tissue, progeny, or part of the citrus tree expresses the NPR1 coding sequence.

13. A plant part of the citrus tree of claim 12, wherein the plant part is selected from the group consisting of fruit, seed, tissue, stem, root, phloem, and flower.

14. A method of increasing tolerance to Huanglongbing in a citrus tree that is otherwise susceptible to Huanglongbing, the method comprising expressing in the citrus tree an NPR1 coding sequence, wherein expression of said NPR1 coding sequence results in increased tolerance to Huanglongbing in the tree.

15. The method of claim 14, wherein the NPR1 coding sequence is from Arabidopsis thaliana.

16. The citrus tree of claim 15, wherein the NPR1 coding sequence comprises a nucleic acid sequence encoding a polypeptide having at least 85% identity to the sequence of SEQ ID NO:21.

17. The citrus tree of claim 16, wherein the NPR1 coding sequence comprises a nucleic acid sequence having at least 85% identity to the sequence of SEQ ID NO:20.

18. The method of claim 14, wherein the citrus tree is a sweet orange tree.

19. The method of claim 18, wherein the sweet orange tree is a ‘Hamlin’ or a ‘Valencia’ sweet orange tree.

20. The method of claim 14, wherein the citrus tree comprises an NPR1 coding sequence operably linked to a heterologous promoter active in plant tissue.

21. The method of claim 20, wherein the heterologous promoter comprises a phloem-specific promoter or a constitutive promoter.

22. The method of claim 21, wherein the phloem-specific promoter comprises an Arabidopsis sucrose-proton symporter 2 (AtSUC2) promoter, or said constitutive promoter comprises a CaMV 35S promoter.

23. A method of increasing yield of a citrus tree comprising expressing in the citrus tree an NPR1 coding sequence, wherein expression of said NPR1 coding sequence results in increased tolerance to Huanglongbing in the tree.

24. The method of claim 23, wherein the NPR1 coding sequence is from Arabidopsis thaliana.

25. The citrus tree of claim 24, wherein the NPR1 coding sequence comprises a nucleic acid sequence encoding a polypeptide having at least 85% identity to the sequence of SEQ ID NO:21.

26. The citrus tree of claim 25, wherein the NPR1 coding sequence comprises a nucleic acid sequence having at least 85% identity to the sequence of SEQ ID NO:20.

27. The method of claim 23, wherein the transgene is operably linked to a heterologous promoter active in plant tissue.

28. The method of claim 27, wherein the heterologous promoter comprises a phloem-specific promoter or a constitutive promoter.

29. The method of claim 28, wherein the phloem-specific promoter comprises an Arabidopsis sucrose-proton symporter 2 (AtSUC2) promoter, or said constitutive promoter comprises a CaMV 35S promoter.

30. A method of producing a citrus tree tolerant to HLB comprising:
(a) introducing into the citrus tree an NPR1 coding sequence; and
(b) reproducing the plant to produce a progeny plant.

31. The method of claim 30, wherein the NPR1 coding sequence is from Arabidopsis thaliana.

32. The citrus tree of claim 31, wherein the NPR1 coding sequence comprises a nucleic acid sequence encoding a polypeptide having at least 85% identity to the sequence of SEQ ID NO:21.

33. The citrus tree of claim 32, wherein the NPR1 coding sequence comprises a nucleic acid sequence having at least 85% identity to the sequence of SEQ ID NO:20.

34. The method of claim 30, wherein step (b) comprises sexual or asexual reproduction of the plant.

35. The method of claim 34, wherein asexual reproduction comprises grafting, budding, layering, or top-working.

36. A citrus tree produced by the method of claim 30.
